(19) United States
(12) Patent Application Publication
(10) Pub. No.: US 2005/0164378 A1
(43) Pub. Date: Jul. 28, 2005

(54) Method for the production of a nerve transplant

(76) Inventor: Wolfgang Schneider, Herford (DE)

Correspondence Address: Bourque & Associates, 835 Hanover Street, Suite 301, Manchester, NH, 03104, US

(21) Appl. No.: 10/503,406

(22) PCT Filed: Aug. 14, 2003
(86) PCT No.: PCT/EP03/01041

(30) Foreign Application Priority Data
Feb. 6, 2002 (DE) 10204966.1

(51) Int. Cl. C12N 5/08

(52) U.S. Cl. 435/366; 424/93.7

(57) ABSTRACT

In a procedure to produce a nerve or other transplantation material, the basal membranes of muscles are colonized with Schwann cells. The colonized basal membrane structure is placed into a nutrient Solution. The Schwann cells are positioned generally continuously along the longitudinal direction of the fibers of the basal membrane over generally the entire length of the implanted material, and the basal membrane thus colonized remains in the nutrient solution until the Schwann cells have aligned themselves generally uniformly and continuously along the longitudinal dimension of the fibers.

### TECHNICAL FIELD

The invention relates to a procedure to produce transplantation material such as nerve transplantation material in which basal membrane from muscles is colonized with Schwann cells, and the colonized basal membrane structure is placed into a nutrient solution for a period of time to allow Schwann cells to align themselves with the basal membrane.

### BACKGROUND INFORMATION

It is known from existing literature that one may colonize basal membranes with Schwann cells to produce nerve transplantation material. A practical procedure to thus effectively colonize basal membranes with Schwann cells is not known, however.

### SUMMARY

The invention includes a method to produce nerve transplantation material in such a fashion that uniform and unrestricted colonization of the basal membrane structure is allowed, and that the prepared nerve transplantation material includes Schwann cells aligned uniformly and continuously along the longitudinal dimension of the fibers.

Schwann cells are inserted continuously along the longitudinal dimension of the fibers of the basal membrane along the entire length of the transplantation material and the basal membrane so colonized remains in the nutrient solution until the Schwann cells have aligned themselves uniformly and continuously along the longitudinal dimension of the fibers.

It is important to note that the present invention is not intended to be limited to a system or method which must satisfy one or more of any stated or implied objects or features of the invention. It is also important to note that the present invention is not limited to the preferred, exemplary, or primary embodiment(s) described herein. Modifications and the invention. It is also important to note that the present invention is not limited to the preferred, exemplary, or primary embodiment(s) described herein. Modifications and substitutions by one of ordinary skill in the art are considered to be within the scope of the present invention, which is not to be limited except by the following claims.

### BRIEF DESCRIPTION OF THE DRAWINGS

FIG. 1 is an illustration of a basal membrane of a muscle.

These and other features and advantages of the present invention will be better understood by reading the following detailed description, taken together with the drawings wherein:

### DETAILED DESCRIPTION OF THE PREFERRED EMBODIMENTS

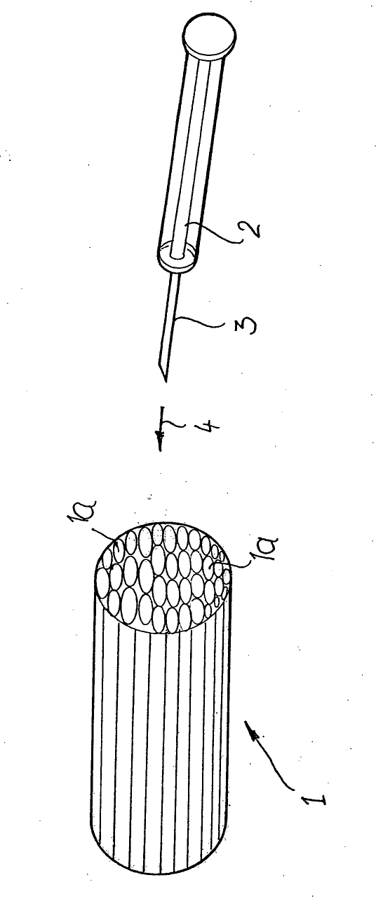

FIG. 1 shows a basal membrane 1 of a muscle. This basal membrane 1 includes a large number of fibers 1a extending along the longitudinal direction of the basal membrane and parallel to one another. A micro-injection needle 3, shown in abbreviated form in the Figure, is placed against the front end of a schematically-represented syringe. The syringe is filled with Schwann cells 2 that may be forced out of the micro-injection needle 3.

To colonize the basal membrane 1, the micro-injection needle 3 containing the Schwann cells 2 is inserted into the basal membrane 1 and pressed into the basal membrane. The Schwann cells are positioned generally continuously along the longitudinal direction of the fibers of the basal membrane over generally the entire length of the implanted material, and the basal membrane thus colonized remains in the nutrient solution until the Schwann cells have aligned themselves generally uniformly and continuously along the longitudinal dimension of the fibers.

In one embodiment, the micro-injection needle 3 containing the Schwann cells 2 is inserted into the basal membrane 1 and pressed into the basal membrane to its left end as shown in the Figure. Subsequently, the syringe is withdrawn to the right white the Schwann cells are carefully simultaneously injected into the basal membrane 1. This procedure is repeated with the basal membrane until the basal membrane is generally filled with Schwann cells 2.

The basal membrane 1 so filled or colonized is subsequently placed into a nutrient medium, where it remains about 72 hours. During this time, the Schwann cells 2 align themselves uniformly and continuously along the longitudinal dimension of the fibers 1a.

During insertion of the Schwann cells 2, a relative motion results between the basal membrane 1 and the micro-injection needle 3 along the longitudinal dimension of these parts. This relative motion thus is in the opposite direction to the exit direction of the Schwann cells 2 from the micro-injection needle 3.

For monitoring purposes, the insertion procedure is performed under a microscope.

As mentioned above, the present invention is not intended to be limited to a system or method which must satisfy one or more of any stated or implied object or feature of the invention and should not be limited to the preferred, exemplary, or primary embodiment(s) described herein. Modifications and substitutions by one of ordinary skill in the art are considered to be within the scope of the present invention, which is not to be limited except by the following claims.

1. A method of producing transplantation material in which the basal membranes of muscles are colonized with Schwann cells, the method comprising the acts of:
inserting Schwann cells generally continuously along a longitudinal dimension of fibers of the basal membrane over generally an entire length of the basal membrane;
placing said colonized basal membrane into a nutrient medium; and
maintaining the colonized basal membrane in the nutrient medium until the Schwann cells have aligned themselves generally uniformly and continuously along the longitudinal dimension of the fibers.

2. The method of claim 1 wherein the colonized basal membrane remains in the nutrient medium for about 72 hours.

3. The method of claim 1 wherein insertion of the Schwann cells into the basal membrane performed by means of a micro-injection needle, and further where in the basal membrane and the injection needle are displaced along the longitudinal direction relative to each other during the insertion process.

4. The method of claim 3 wherein the relative displacement between the basal membrane and the injection needle is in an opposite direction relative to the exit direction of the Schwann cells from the micro-injection needle.

5. The method of claim 1 wherein the insertion of Schwann cells is performed under a microscope 6. The method of claim 1 wherein the uniform positioning of Schwann cells occurs across generally an entire cross-section of the basal membrane.

6. The method of claim 1 wherein the transplantation material produced by the method includes nerve transplantation material.
